(19) United States
(12) Patent Application Publication
(10) Pub. No.: US 2025/0152595 A1
(43) Pub. Date: May 15, 2025

(54) MEDICATION TO ALLEVIATE NAUSEA WITH OR WITHOUT VOMITING, VERTIGO, AND PAIN

(71) Applicant: Lisa Myslinski, Chicago, IL (US)

(72) Inventor: Lisa Myslinski, Chicago, IL (US)

(21) Appl. No.: 18/505,834

(22) Filed: Nov. 9, 2023

(51) Int. Cl. A61K 31/522; A61K 31/138; A61K 31/167; A61K 31/192; A61K 31/495; A61K 31/616

(52) U.S. Cl. CPC A61K 31/522; A61K 31/138; A61K 31/167; A61K 31/192; A61K 31/495; A61K 31/616

(57) ABSTRACT

This invention is new because no prior art discusses using any of these antiemetics as a single entity medication or in combination with any analgesic(s) for the indication of nausea with or without vomiting; vertigo including MAV, photophobia, phonophobia, with or without pain. This invention also differs from prior art in that the aforementioned antiemetics in any combination with the aforementioned analgesic(s), are also able to combined with or without a nasal decongestant dextromethorphan guaifenesin. The interchangeability of the drugs and doses allows for versatility which prior art does not offer. Actual dosages used will be within that range at therapeutic, efficacious, and safe dosages in subjects for the treatment of the indication(s) being claimed and/or exhibited by subjects. Utilizing subtherapeutic and supratherapeutic doses in the ranges are not obvious or usual doses utilized by people knowledgeable in the prior art, differentiating it from prior art and supporting patentability.

### BACKGROUND OF THE INVENTION

Nausea with or without vomiting, vertigo, and pain are common symptoms associated with many medical conditions. These medical conditions may include viruses such as norovirus or influenza, migraines, headaches, menstrual symptoms, or any condition in which these symptoms may be present. Norovirus causes 19 to 21 million cases each year of acute gastroenteritis in the United States. There were 31 million cases of influenza in the United States and 1 billion worldwide.

Nausea, vomiting, and pain occur as major symptoms during menstruation. In comparison with patients who had a regular menstrual cycle, an irregular menstrual cycle increased the incidence of nausea or vomiting from 20.5% to 40.5%.

Approximately 38 million people in the United States suffer from migraines. About 73% of migraineurs suffer from migraine associated nausea. About 40% of migraineurs suffer from migraine associated vertigo.

Problems with the prior art is that nausea with or without vomiting, vertigo, and pain are not completely addressed in currently available over-the-counter medications. Some prior art may mention the use of some of the antihistamines or antiemetics used in this invention, but are not specified for nausea with or without the other symptoms for any medical condition(s) in which these symptoms are exhibited. This patent addresses coverage of these symptoms; nausea with or without vomiting, vertigo, phonophobia, photophobia, and with or without pain; with or without nasal congestion; with or without cough; with or without chest congestion; for any medical condition(s) that warrants the use of these medications for these symptoms. Consumers should have a comprehensive medication that addresses nausea with or without vomiting, vertigo, and pain and/or any of the aforementioned symptoms for any medical condition(s) in which an antiemetic would be warranted or helpful. This invention is different than prior art and is an improvement to prior art in that it allows for the treatment of the singular symptoms or any combination of symptoms using any combination of the medications in this invention by using them as single entity medications; such as allowing the usage of diphenhydramine for nausea only, or a combination(s) of medications such as diphenhydramine with acetaminophen for nausea and pain; meclizine with any analgesic such as acetaminophen, ibuprofen, or naproxen for example. This patent is different from prior art in that in order to effectively to treat viruses such as influenza or the stomach flu for example, this patent allows any antiemetic such as meclizine, to be combined in any manner with an analgesic, a nasal decongestant such as phenylephrine including any newly reformulated versions of phenylephrine or pseudoephedrine, a cough suppressant such as dextromethorphan hydrobromide or dextromethorphan polistirex, and the expectorant guaifenesin. The reference of combination in any manner refers to the interchangeability of the drugs; such as meclizine with an analgesic and phenylephrine for example, or the combination of an antiemetic such as meclizine with an analgesic and a nasal decongestant and a cough suppressant and an expectorant, which prior art does not address.

### BRIEF SUMMARY OF THE INVENTION

My invention consists of a medication to treat nausea with or without vomiting, vertigo, phonophobia, photophobia, and with or without pain for any medical condition in which these symptoms occur. These medical conditions can include the aforementioned symptoms associated with the flu, menstruation, headaches or migraines. The medications in my invention consist of an anti-emetic with or without an analgesic(s) for the treatment of nausea with or without vomiting, vertigo, and with or without pain; with or without nasal congestion; with or without cough; with or without chest congestion. The anti-emetics included are diphenhydramine, meclizine, dimenhydrinate, cyclizine, betahistine, cinnarizine, promethazine; metoclopramide; prochlorperazine; and the antiemetic ondansetron which is a serotonin 5-HT3 receptor antagonist; with or without an analgesic(s) such as acetaminophen with or without caffeine and with or without aspirin, acetaminophen as a single entity by itself; acetaminophen in combination with ibuprofen; acetaminophen in combination with naproxen; aspirin; ibuprofen; naproxen; ketorolac; with or without a nasal decongestant phenylephrine including any reformulated version of phenylephrine or pseudoephedrine; with or without a cough suppressant such as dextromethorphan hydrobromide or dextromethorphan polistirix; with or without an expectorant guaifenesin.

The anticholinergic properties of these antihistamines or antiemetics mentioned above helps prevent nausea and vomiting; metoclopramide prevents nausea by blocking dopamine D2 and serotonin 5-HT3 receptors; prochlorperazine blocks dopamine D2 receptors; and the antiemetic ondansetron prevents nausea and vomiting by blocking the action of serotonin. Acetaminophen, aspirin, and the nonsteroidal medications are analgesics which relieve pain. Caffeine accelerates absorption and enhances the analgesic effect of acetaminophen. Phenylephrine and pseudoephedrine are nasal decongestants used to alleviate a stuffy nose, dextromethorphan hydrobromide and dextromethorphan polistirex are cough suppressants, and guaifenesin thins mucous or phlegm in the air passages and is an expectorant.
(1) This invention improves upon prior art because it includes a broad range of antihistamines or antiemetics being utilized for their antiemetic effects in combination with or without a broad range of analgesics including, but not limited to, acetaminophen with or without caffeine and aspirin; acetaminophen as a single entity medication; acetaminophen in combination with ibuprofen; acetaminophen in combination with naproxen; aspirin; ibuprofen; naproxen; ketorolac with or without a nasal decongestant phenylephrine including any reformulated version of phenylephrine and pseudoephedrine; with or without dextromethorphan hydrobromide or dextromethorphan polistirex; with or without guaifenesin. Prior art may include only one specific antihistamine or antiemetic with an analgesic for a specific indication. This invention involves treatment of nausea with or without vomiting, vertigo, and with or without pain for any medical condition such as influenza, viruses, menstruation, headaches and migraines (not all-inclusive) that this combination proves efficacious for treating the aforementioned symptoms. Hence, this invention provides broader coverage of symptoms and treatment of symptoms and the ability to utilize a combination of any of the antiemetics with or without any of the analgesics mentioned above, either as single entity as in any combination(s) for effective treatment of the symptoms exhibited. For example, this invention allows one to combine diphenhydramine with acetaminophen and with or without caffeine as well as with or without aspirin. One may combine diphenhydramine or meclizine, or dimenhydrinate with acetaminophen or ibuprofen or naproxen. The antiemetics in this invention can be used alone as single entity medications for the treatment of symptom(s) mentioned previously. For example, diphenhydramine can be used for nausea. Finally, this invention offers versatility of medication combinations which may be important for patients with contraindications to certain medications. For instance, a person with liver failure would not take acetaminophen. The option to use diphenhydramine or meclizine with ibuprofen enables another option for a patient with such a contraindication as described in the prior example. For example, this medication can combine diphenhydramine with acetaminophen, caffeine, aspirin or any combination thereof to treat any medical condition, in which said subject, is experiencing nausea either with or without vomiting, vertigo including migraine-associated vertigo or photophobia or phonophobia, and with or without pain. Another example, diphenhydramine can be combined with ibuprofen to alleviate any or all or any combination of the symptoms mentioned within this claim.

### DETAILED DESCRIPTION OF THE INVENTION

This invention contains the antihistamines or antiemetics diphenhydramine, meclizine, dimenhydrinate, cyclizine, betahistine, cinnarizine, promethazine; metoclopramide; prochlorperazine; and ondansetron with an analgesic including acetaminophen with or without caffeine or aspirin or any combination thereof, acetaminophen as a single entity medication, acetaminophen in combination with ibuprofen, aspirin, ibuprofen, and naproxen, ketorolac; with or without a nasal decongestant phenylephrine or any reformulated version of phenylephrine and pseudoephedrine; with or without a cough suppressant dextromethorphan hydrobromide or dextromethorphan polistirex; with or without a chest decongestant guaifenesin; used interchangeably in different combinations to alleviate symptoms of nausea with or without vomiting, vertigo, photophobia, phonophobia, and pain for any medical condition or disease in which one exhibits these symptoms such as influenza, viruses, headaches, migraines, or menstruation. These examples of medical conditions are not all-inclusive. For instance, if a clinician sees fit to prescribe any combination of these medications in this invention for treatment for any or all the symptoms in this invention, such as post-operation nausea or nausea associated with morning sickness in pregnancy; then this would be allowed or available to treat that particular indication at an appropriate, effective dose for that indication.

The antihistamines used in this invention have anticholinergic properties which helps prevent nausea and vertigo. Metoclopramide, prochlorperazine and ondansetron are antiemetics. The analgesics in this invention relieve pain. Caffeine enhances the analgesic effect of acetaminophen. Ergot alkaloids treat migraines by constricting blood vessels, blocking pain signals, and reducing inflammation. Triptans have a vasoconstrictive effect which helps reduce migraine headaches. Magnesium may help treat migraines by blocking brain signals that cause migraines and may also block pain-transmitting chemicals in the brain. Phenylephrine and pseudoephedrine are nasal decongestants to relieve stuffy nose. Dextromethorphan hydrobromide and dextromethorphan polistirex are cough suppressants. Guaifenesin is an expectorant to thin mucous or phlegm in the bronchial passageways.

The symptoms that are covered in this invention include nausea with or without vomiting, vertigo including migraine-associated vertigo, phonophobia, photophobia, and/or pain; with or without nasal congestion or stuffy nose; with or without cough; with or without chest congestion or mucous in the bronchial passageways. This invention can be used if a person exhibits any or all of these symptoms. For instance, if a person has only nausea, then this invention can be used. If a person has nausea with vertigo, then this product can be used. If a person has nausea with a headache or a migraine, nausea, nasal congestion or stuffy nose, a cough, and chest congestion, or any combination of these symptoms, then this invention can be used. If a person exhibits all multiple symptoms such as pain, nausea and vertigo, this invention or product can be used utilizing any combination(s) of the medications included in the claims or as single entity medications. This invention is not all inclusive for usage or combination of symptoms or medications in that it may be used according to the symptoms exhibited by said subject; for example, if a person has the stomach flu with nausea, headache, fever, nasal congestion, cough, and chest congestion, then all the above aforementioned drugs may be used together to treat those symptoms such as meclizine, acetaminophen, phenylephrine, dextromethorphan hydrobromide, and guaifenesin. This invention is different and an improvement to prior art in that it allows the first product to treat nausea along with all other possible flu symptoms, because prior art does not include an antiemetic in available multi-symptom flu products.

This invention offers users and/or clinician's options of usage for different symptoms and combinations of medications. For example, one may use acetaminophen or ibuprofen, or naproxen, or acetaminophen in combination with ibuprofen, acetaminophen in combination with naproxen, in combination with any of the antiemetics listed in this invention. Ibuprofen with meclizine is one example of a possible combination. Acetaminophen with caffeine and aspirin in combination with diphenhydramine is an additional example of how these medications can offer different options of combinations for treatment of these symptoms. The ability to use different combinations of these medications differentiate it from prior art by offering interchangeability as well as the ability to treat single symptoms such as nausea and/or the ability to treat a multi-symptom indication such as in migraines in which case, one may exhibit a multitude of symptoms such as pain, nausea, photophobia, phonophobia, and vertigo.

Diphenhydramine is currently available over-the-counter in the following strengths; (1) Liquid for 12.5 mg per 5ml, (2) 25 mg tablet, (3) 25 mg tablet. Meclizine is currently available over-the-counter in the following strengths (1) 12.5 mg tablet, (2) 25 mg tablet, (3) 50 mg tablet. Dimenhydrinate is currently available over-the-counter in the following strengths: (1) 25 mg tablet, and (2) 50 mg tablet. Cyclizine is available as 50 mg tablets and 50 mg injections. Betahistine is available in 8 mg and 16 mg tablets. Cinnarizine is available in 15 mg. 25 mg, and 75 mg tablets. Promethazine comes in 25 mg/ml and 50 mg/ml injections, and in 12.5 mg. 25 mg, and 50 mg tablet strengths. Metoclopramide is available as a 5 mg/ml injectable form; in 5 mg/5ml and 10 mg/10ml oral solutions; and in 5 mg and 10 mg tablets. Prochlorperazine is available as injectable in 10 mg/2ml and 50 mg/10ml and comes in 5 mg and 10 mg tablets; and ondansetron is available in a 4 mg/2ml injectable, a 4 mg/5ml oral solution, and in 4 mg. 8 mg. and 24 mg oral tablets. Cyclizine is dosed at 50 mg up to three times a day, up to 200 mg/day. Betahistine dose ranges from 24 mg to 48 mg per day administered two to three times daily. Cinnarizine is dosed at 25 mg to 75 mg per day in divided doses. Promethazine dosing for nausea and vomiting is 12.5 mg to 25 mg every four to six hours if needed. Metoclopramide is dosed at 10 to 15 mg up to four times a day for nausea. Prochlorperazine is dosed at 5 to 10 mg orally every 6 to 8 hours. Ondansetron is dosed at 4 mg to 8 mg three times a day for nausea and vomiting. The nasal decongestant phenylephrine is dosed at 10 mg every four hours as needed not to exceed 60 mg in 24 hours and pseudoephedrine is dosed at 60 mg every four to six hours. Dextromethorphan hydrobromide is dosed at 15 mg to 30 mg three to four times daily and dextromethorphan polistirex is dosed at 10 mg to 20 mg every four hours as needed or 30 mg every six to eight hours as needed. Guaifenesin is an expectorant and is dosed at 200 mg to 400 mg every four hours and the extended-release formulations contain either 600 mg or 1200 mg dosed twice daily.

The available dosage forms may include a liquid form such as a solution or suspension, or a solid oral dosage form such as a capsule, caplet, or tablet, and/or an oral chewable form, orally disintegrating (ODT) tablets, dissolving strip, powder, effervescent powder or tablets, sublingual tablets; transdermal; intravenous; intramuscular; intradermal; subcutaneous; or any form that goes onto or into the skin via a device or patch that can be available either as prescription or nonprescription. The strengths of the active ingredients can vary in a dosage range that is considered subtherapeutic or supratherapeutic from the current available strengths of each medication according to the strengths that are currently available over-the-counter commercially. For example, diphenhydramine in tablet form is currently available as a 12.5 mg tablet/capsule/solution, a 25 mg tablet/capsule and a 50 mg tablet/capsule. This invention allows for a dosage range utilizing strengths that are below the lowest available strength and above the highest strength commercially available which includes all strengths in-between including current commercially available strengths. In other words, this invention for diphenhydramine would cover strengths from about 1.25 mg to 90 mg. These strengths are not considered usual therapeutic or effective strengths which would be utilized by a person with knowledge in the prior arts. This makes it different from prior art and, therefore patentable because these doses would not be used by a person knowledgeable in the prior art. Additionally, the ability to combine the antiemetics, analgesics, nasal decongestants, cough suppressants, and chest decongestant in any combination and interchangeably also makes this patent different from prior art in that prior art does not exist utilizing these possible combinations allowing this patent to treat symptoms and condition(s) accordingly.

The dosages for the analgesics in this invention are commonly known and listed in literature for the treatment of pain. Ibuprofen is available over-the-counter in a 200 mg tablet form and as a liquid suspension. Ibuprofen is commercially available over-the-counter as a 200 mg tablet/capsule and a 125 mg tablet in combination with acetaminophen 250 mg solid oral dosage form. Naproxen is available in a dose of 220 mg dose over-the-counter commercially. Ketorolac is available as injection in 15 mg/ml and 30 mg/ml; as 30 mg/ml intramuscular; and in 10 mg oral tablets. Acetaminophen is commercially available alone and in combination with caffeine, aspirin; and ibuprofen as a 250 mg strength. Acetaminophen is available as a single entity strength in 325 mg, 500 mg, and 650 mg solid oral dosage forms and some liquid formulations. Again, ranges of available strengths for each of these analgesics will allow for doses lower than the lowest available dose commercially available and higher than the highest strength currently available commercially or over-the-counter including all strengths in-between. This invention may be available as an over-the-counter medication or as a prescription medication. For drugs not currently available in the United States, such as cyclizine, betahistine, and cinnarizine, these drugs would follow the guidelines per each country's governing drug regulations. Dosage guidelines for the allowed drugs in the United States would be per current Food and Drug Administration dosing guidelines for each medication(s) for each indication(s). For example, a normal analgesic dose of acetaminophen is 1 gram every 6 hours as needed for pain, not to exceed 4 grams per day as a total daily dose. Therefore, even though the allowable range in this invention covers a range of strengths for acetaminophen from 25 mg to 975 mg; the proper safe and effective dosage for the treatment of pain would be used for human safety and efficacy.

Contraindications and precautions and dosages for each indication (i.e., nausea, vertigo, analgesia) for each medication in this invention will follow all applicable guidelines and dosages set forth by the Food and Drug Administration for provision of safety and efficacy in humans. The medications should be stored at approximately 20-25 degrees Celsius (68-77 degrees Fahrenheit) and avoid humidity. Some products that may require refrigeration would be stored at 36-46 degrees Fahrenheit (2-8 degrees Celsius). Labeling would include to keep out of reach of children and close cap tightly after each use. This invention will most likely be used in people 12 years of age and older. The chewable and liquid formulations may be beneficial for pediatric populations if studies performed prove beneficial and efficacious for that population, leaving this invention open for usage in the pediatric population per proper, safe and effective dosages.

This invention will also contain inactive ingredients such as fillers and other commonly used inactive ingredients in caplet and tablet manufacturing such as gelatin; hydroxypropyl cellulose; titanium dioxide; etc. The aforementioned list are just examples of inactive ingredients that are frequently used in caplet/tablet/capsule formulation and found in commonly in literature about medication manufacturing. The inactive ingredients commonly used in liquid formulations are listed in literature as well.

Manufacturing of medications listed in this invention would utilize the tools or manufacturing equipment that is customarily used in the pharmaceutical industry per good manufacturing practices according to Food and Drug Administration guidelines. Drugs not currently approved or available in the United States would follow the corresponding country's drug regulations.

1) A medication to treat or alleviate symptoms, singularly or any combination of symptoms, of nausea, with or without vomiting, vertigo including migraine-associated vertigo, photophobia, phonophobia, and with or without pain associated with any medical condition in which these symptoms are exhibited, comprising any and all of the antihistamines or antiemetics including but not limited to diphenhydramine, meclizine, dimenhydrinate cyclizine, betahistine, cinnarizine, promethazine, metoclopramide, prochlorperazine, and ondansetron used as single entity or in any combination with any analgesic(s), prescription or nonprescription, including but not limited to acetaminophen with or without caffeine and with or without aspirin and in any combination thereof such as acetaminophen with aspirin and caffeine or acetaminophen with aspirin or acetaminophen with caffeine or acetaminophen alone as a single entity medication; acetaminophen with ibuprofen; acetaminophen with naproxen; aspirin; ibuprofen; naproxen; ketorolac; comprising of any and all of the antiemetics including, but not limited to: diphenhydramine at a dosage of from about 1.25 mg to about 90 mg; meclizine at a dosage of from about 1 mg to about 95 mg; dimenhydrinate at a dosage of from about 2.5 mg to about 95 mg; cyclizine at a dosage of from about 10 mg to about 200 mg; betahistine at a dosage of from about 5 mg to about 60 mg; cinnarizine at a dosage of from about 10 mg to about 80 mg; promethazine at a dosage of from about 3 mg to about 100 mg; metoclopramide at a dosage of from about 2 mg to about 80 mg; prochlorperazine at a dosage of from about 2.5 mg to about 80 mg; ondansetron at a dosage of from about 1 mg to about 20 mg; alone as a single entity or in combination with any analgesic(s) including, but not limited to: acetaminophen at a dosage of from about 25 mg to about 975 mg as a single entity or in combination with or without aspirin at a dosage of from about 8 mg to about 617 mg and with or without caffeine at a dosage of from about 6 mg to about 190 mg and in any combination thereof; acetaminophen at a dosage of from about 25 mg to about 975 mg in combination with ibuprofen at a dosage of from about 12.5 mg to about 800 mg; acetaminophen at a dosage of from about 25 mg to about 975 mg in combination with naproxen at a dosage of from about 22 mg to about 1000 mg; aspirin at a dosage of from about 8 mg to about 617 mg; ibuprofen at a dosage of from about 12.5 mg to about 800 mg; and naproxen at a dosage of from about 22 mg to about 1000 mg; ketorolac at a dosage of from about 2.5 mg to about 120 mg; with the terminology, “dosage of from about” defined as or referring to the range of a single dose or any single dose within that dosage range given at a single moment of time which will be given at safe and efficacious dosing intervals for the medications utilized such as every 6-8 hours for example, and does not refer in any manner to a range of the total daily dose or dosage.

2) A drug according to claim 1, comprising of the allowable addition of any vitamin(s) and/or mineral(s) including, but not limited to vitamin B2, zinc, and magnesium; natural ingredients including, but not limited to ginger, chamomile, and feverfew; and/or electrolytes which can be incorporated into any dosage form or any combination of drugs according to claim 1 such as an effervescent tablet or oral powder dosage form as examples or packaged separately including co-packaged within the medication packaging in its own individual packets or packaging.

3) A method of treating the symptoms, according to claim 1, of nausea with or without vomiting; vertigo including migraine-associated vertigo; photophobia; phonophobia; and with or without pain in any medical condition in which these symptoms are exhibited, including but not limited to migraine, headaches, influenza, viruses, menstruation; utilizing the medication(s), comprising any combination of the medications at effective dose(s) per indication(s); administering to a human subject in need thereof, comprising administering to said human subject either as a single entity medication or any combination(s) thereof of medications.

4) A medication comprising interchangeable combinations of drugs according to claim 1, comprising the different variations and combinations as either single entity medications or interchangeable combinations of claimed medications.

5) A dosage, according to claim 1, comprising an efficacious and safe dose(s) for treatment of the symptoms and/or medical indication(s) exhibited by said subject.

6) A medication, according to claim 1, that can be manufactured in any form(s) such as a solid oral dosage form(s) including tablets of any form including but not limited to orally disintegrating tablets (ODT), gel-tabs, sublingual tablets, chewable tablets, powdered tablets; effervescent powder or tablets, regular compressed tablets, lozenge, a dissolving strip, caplets, capsules, gelcaps, liquid-filled capsules, capsules and/or tablets as immediate release or extended-release formulations; liquid form(s) such as solutions, suspensions including extended-release (long-acting) suspensions, elixirs; powders for any route of administration; oral; intranasal; rectal; intravenous; intramuscular; intradermal; subcutaneous; transdermal; or any form that goes onto (ie. transdermal patch) or into the skin via a device that can be available either as prescription and/or nonprescription also considered as over-the-counter status.

7) A medication according to claim 1, that can be combined and used interchangeably in any combination with the following medications, utilizing any dosage form and any route of administration: with or without any ergot alkaloid such as dihydroergotamine at a dosage of from about 0.5 mg to about 80 mg; with or without any triptan or selective serotonin receptor agonist such as sumatriptan at a dosage of from about 10 mg to about 400 mg; with or without magnesium in any salt form (ie. magnesium oxide, magnesium sulfate, etc.) at a dosage of from about 100 mg to about 4,000 mg.

8) A medication to treat or alleviate symptoms, singularly or any combination of symptoms, of nausea, with or without vomiting, vertigo including migraine-associated vertigo, photophobia, phonophobia, and with or without pain, fever, body aches, headache, migraine; with or without nasal congestion or stuffy nose; with or without any type of cough (ie. productive or nonproductive); with or without chest congestion or mucous secretions also known as phlegm in the bronchial passageways; with or without rhinorrhea commonly called runny nose, associated with any medical condition(s) in which these symptom(s) are exhibited, comprising any and all of the antihistamines or antiemetics including but not limited to diphenhydramine, meclizine, dimenhydrinate, cyclizine, betahistine, cinnarizine, promethazine, metoclopramide; prochlorperazine; and ondansetron used as single entity or in any combination with any analgesic(s), prescription or nonprescription, including but not limited to acetaminophen with or without caffeine and with or without aspirin and in any combination thereof such as acetaminophen with aspirin and caffeine or acetaminophen with aspirin or acetaminophen with caffeine or acetaminophen alone as a single entity medication; acetaminophen with ibuprofen; acetaminophen with naproxen; aspirin; ibuprofen; naproxen; ketorolac; with any and all of the above aforementioned drugs used in any combination with the nasal decongestants pseudoephedrine, phenylephrine including any newly reformulated version of phenylephrine; with or without and in any combination with the cough suppressant dextromethorphan hydrobromide, dextromethorphan polistirex; with or without and in any combination with the chest decongestant or expectorant guaifenesin; comprising of any and all of the antiemetics: diphenhydramine at a dosage of from about 1.25 mg to about 90 mg; meclizine at a dosage of from about 1 mg to about 95 mg; dimenhydrinate at a dosage of from about 2.5 mg to about 95 mg; cyclizine at a dosage of from about 10 mg to about 200 mg; betahistine at a dosage of from about 5 mg to about 60 mg; cinnarizine at a dosage of from about 10 mg to about 80 mg; promethazine at a dosage of from about 3 mg to about 100 mg; metoclopramide at a dosage of from about 2 mg to about 80 mg; prochlorperazine at a dosage of from about 2.5 mg to about 80 mg; ondansetron at a dosage of from about 1 mg to about 20 mg; alone as a single entity or in combination with any analgesic(s) including, but not limited to: acetaminophen at a dosage of from about 25 mg to about 975 mg as a single entity or in combination with or without aspirin at a dosage of from about 8 mg to about 617 mg and with or without caffeine at a dosage of from about 6 mg to about 190 mg and in any combination thereof; acetaminophen at a dosage of from about 25 mg to about 975 mg in combination with ibuprofen at a dosage of from about 12.5 mg to about 800 mg; acetaminophen at a dosage of from about 25 mg to about 975 mg in combination with naproxen at a dosage of from about 22 mg to about 1000 mg; aspirin at a dosage of from in about 8 mg to about 617 mg; ibuprofen at a dosage of from about 12.5 mg to about 800 mg; naproxen at a dosage of from about 22 mg to about 1000 mg; ketorolac at a dosage of from about 2.5 mg to about 120 mg; with or without and in any combination with pseudoephedrine at a dosage of from about 10 mg to about 300 mg, phenylephrine or any newly reformulated version of phenylephrine at a dosage of from about 10 mg to about 60 mg; with or without and in any combination with dextromethorphan hydrobromide or any form at a dosage of from about 10 mg to about 200 mg, dextromethorphan polistirex at a dosage of from about 10 mg to about 200 mg; with or without and in any combination with guaifenesin at a dosage of from about 5 mg to about 3,000 mg; with all of the above aforementioned drug(s) used as single entity, used interchangeably, or in any combination with each other to treat or alleviate the symptoms exhibited by said subject; with the terminology, “dosage of from about” defined as or referring to the range of a single dose or any single dose within that dosage range given at a single moment of time which will be given at safe and efficacious dosing intervals for the medications utilized such as every 6-8 hours for example, and does not refer in any manner to a range of the total daily dose or dosage.

9) A drug according to claim 8, comprising of the allowable addition of any vitamin(s) and/or mineral(s) including, but not limited to vitamin C, zinc, and magnesium; natural ingredients including, but not limited to ginger, chamomile, or feverfew; and/or electrolytes which can be incorporated into any dosage form such as an effervescent tablet or oral powder dosage form as examples or packaged separately including co-packaged within the medication packaging in its own individual packets or packaging.

10) A method of treating the symptoms, according to claim 8, of nausea with or without vomiting; vertigo including migraine-associated vertigo; photophobia; phonophobia, sneezing; and with or without pain, body aches, fever, headache, migraine; and with or without nasal congestion or stuffy nose, head congestion, sinus congestion; and with or without cough either productive or nonproductive; and with or without chest congestion causing phlegm or mucous in the bronchial passageways; in any medical condition(s) in which these symptoms are exhibited, including but not limited to migraine, headaches, influenza, stomach flu, viruses, menstruation; pain and body aches, fever, nasal congestion or stuffy nose, sinus congestion, head congestion associated with the flu, hay fever, allergic rhinitis, any allergic condition(s) such as but not limited to allergic sinusitis; common cold; utilizing the medication(s); comprising any combination of the medication(s) for treatment of any combination(s) of symptoms exhibited at effective dose(s) per indication(s); administering to said subject in need thereof. comprising administering to said subject either as a single entity medication or any combination(s) thereof of medications.

11) A medication comprising interchangeable combinations of drugs according to claim 8, comprising the different variations and combinations as either single entity medications or interchangeable combinations of claimed medications.

12) A dosage, according to claim 8, comprising an efficacious and safe dose(s) for treatment of the symptoms and/or medical indication(s) exhibited by said subject.

13) A medication, according to claim 8, that can be manufactured in any form(s) such as a solid oral dosage form(s) including tablets of any form including but not limited to orally disintegrating tablets (ODT), gel-tabs, sublingual tablets, chewable tablets, powdered tablets; effervescent powder or tablets, regular compressed tablets, lozenge, a dissolving strip, caplets, capsules, gelcaps, liquid-filled capsules, capsules and/or tablets as immediate release or extended-release formulations; liquid form(s) such as solutions, suspensions including extended-release (long-acting) suspensions, elixirs; powders for any route of administration; oral; intranasal; rectal; intravenous; intramuscular; intradermal; subcutaneous; transdermal; or any form that goes onto (ie. transdermal patch) or into the skin via a device that can be available either as prescription and/or nonprescription also considered as over-the-counter status.
